(19) United States
(12) Patent Application Publication
(10) Pub. No.: US 2007/0135790 A1
(43) Pub. Date: Jun. 14, 2007

(54) MOBILE PHONE MEDICAMENT CONTAINER AND COMMUNICATION SYSTEM

(76) Inventor: DAVID M. AUERBACH, Calabasas, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/679,322

(22) Filed: Feb. 27, 2007

Related U.S. Application Data
(63) Continuation-in-part of application No. 10/910,129, filed on Aug. 2, 2004, now abandoned.

(51) Int. Cl. A61M 31/00

(52) U.S. Cl. 604/500; 604/65

(57) ABSTRACT

An apparatus including a compartment to store a medicament, a communication facility to exchange data with a health information provider, a display system to present information to a user, and a timekeeping subsystem to activate the display at a predetermined time is described and claimed. Methods of using similar devices, and computer software to control such devices, are also described and claimed.

### CROSS-REFERENCE TO RELATED APPLICATION

This application is a continuation-in-part of co-pending U.S. patent application Ser. No. 10/910,129, filed Aug. 2, 2004.

### FIELD

Medicament containers.

### BACKGROUND

Prior art devices and methods have been developed which inform patients that it is time to take a medicament. For example, U.S. Pat. No. 6,507,275 recites a pill dispensing case including a reminder for assisting a patient to remember to take one or more pills at a preferred time.

An embodiment includes a microprocessor held in a case. The microprocessor includes a 24 hour clock and outputs information to both a warning indicator and a data display window at programmed times. The microprocessor is connected to a data link interface which is capable of receiving programming information. The case may include one or more pill compartments depending on whether there is more than one type of medicament. A warning signal transmission unit may also be provided separately from the case and may transmit a warning signal generated from the case to a mobile or stationary receiver via wireless or wired output.

### BRIEF DESCRIPTION OF DRAWINGS

Embodiments are illustrated by way of example and not by way of limitation in the figures of the accompanying drawings in which like references indicate similar elements. It should be noted that references to “an” or “one” embodiment in this disclosure are not necessarily to the same embodiment, and such references mean “at least one.”

FIG. 1 shows a top, side perspective view of one embodiment of a medicament container.

FIG. 2 shows a front, side perspective view of the medicament container of FIG. 1.

FIG. 3 shows the medicament container of FIG. 1 being carried on a person.

FIG. 4 shows a cross-sectional view of the medicament container of FIG. 1 including a schematic view of representative electronic components associated with the medicament container and shows the medicament container connected to an independent computing device.

FIG. 5 shows a representation of a series of displays of the medicament container of FIG. 1.

FIG. 6 shows an embodiment of a method instructing a patient to take medicaments.

FIG. 7 shows a top, side perspective view of another embodiment of a medicament container that is a mobile communication and computation apparatus such as a cellular telephone and/or personal digital assistant.

FIG. 8 shows a schematic view of a communication system including the mobile communication and computation device of FIG. 7.

FIG. 9 shows an embodiment of a method instructing a patient to take a medicament through a mobile communication and computation device.

FIG. 10 shows an embodiment of a method for a facility to instruct a patient to take a medicament through a telecommunication device.

FIG. 11 shows an embodiment of a method where a user communicates with a communication facility to establish a notifying system for taking a medicament.

### DETAILED DESCRIPTION

It is common for patients to take multiple medicaments for multiple ailments during the same period of time. Also, it is common for patients to forget to take a medicament at times instructed by a healthcare professional. In situations involving different medicaments, when patients do remember, patients often forget how each medicament looks individually. This is particularly true with prescription medicaments since most prescription medicaments rely on the package label to identify the medicament.

These problems are amplified when a patient is scheduled to take a medicament at a time when the patient is away from home and the patient's attention is directed toward something other than taking a medicament. Patients typically do not take an entire package (e.g., a pill bottle), which identifies the medicament, with them when going somewhere. Rather, patients tend to just carry (e.g., in a container) the minimum amount of medicament(s) necessary to be taken during their absence from home. Additionally, while away from home many patients become distracted and forget to take their medicament(s) at the scheduled time.

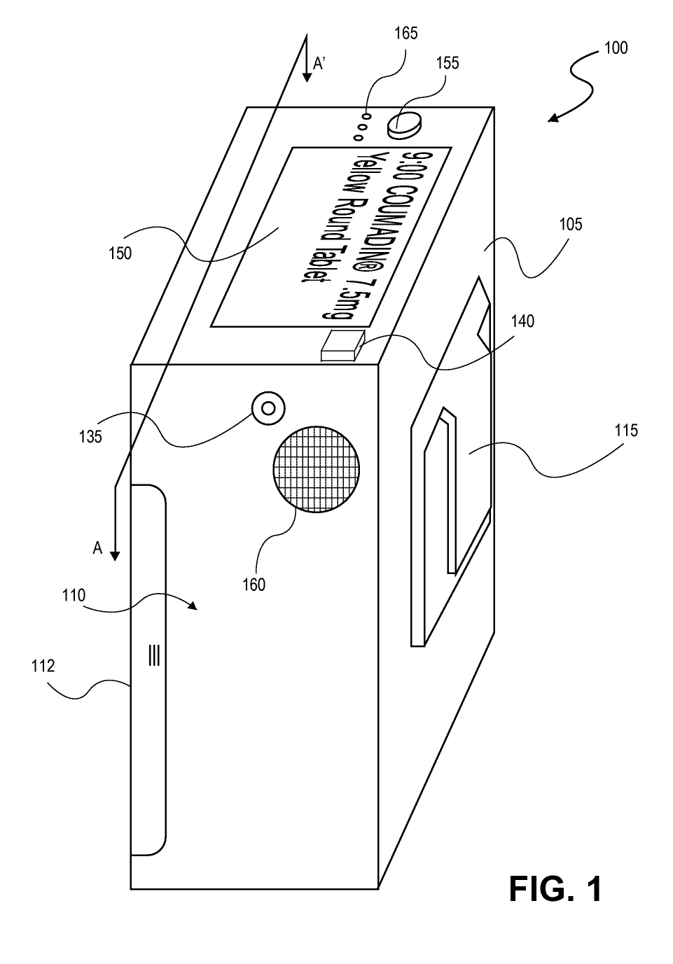

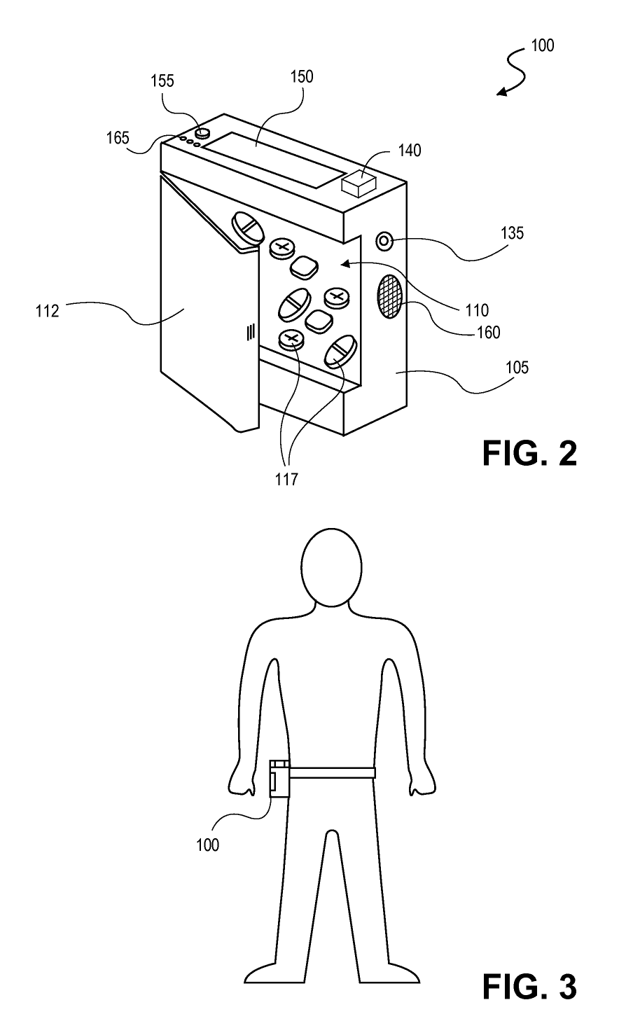

FIG. 1 and FIG. 2 show an embodiment of a medicament container. Representatively, medicament container 100 includes case 105, which includes enclosed compartment 110 of a size suitable to hold several medicaments. In one embodiment, compartment 110 is unrestricted in that it does not contain any sub-compartments (e.g., walls) for separating medicaments. Suitable medicaments include, but are not limited to tablets, capsules, gel capsules, liquids, powders, sprays, lotions, creams, ointments, etc. containing a therapeutic compound or portion thereof.

By “therapeutic compound” it is meant any substance used to treat (including prevent, diagnose, alleviate, or cure) a malady, affliction, nutritional deficiency, disease or injury in a patient. The term “therapeutic compound” is also meant to include substances that are a supplement for improving the nutritional, physical, or emotional well-being of a patient such as vitamins, minerals, or herbal based supplements. By “patient” it is meant a human and/or animal such as a mammal or reptile under the care of an administrator such as a physician or veterinarian.

Case 105 can be made of plastic, metal or any other durable, light-weight material suitable for use in holding medicaments and transportable by a patient. In one embodiment, case 105 is in the shape of a rectangular (including a square) box. In terms of plastic material, case 105 may be formed through molding techniques. Although shown as a rectangular box, it is contemplated that case 105 may have any shape suitable for use in holding medicaments and equipment suitable for alerting a patient it is time to take a medicament. For example, in another embodiment, case 105 may include a nylon or cloth body defining compartment 110 (e.g., of a size similar to a purse or “fanny pack” on a belt or to be worn around a patient's waist).

As shown in FIG. 1 and FIG. 2, case 105 includes a top portion, a bottom portion, a front portion, a back portion, and opposing side portions that define a depth of case 105. Representatively, case 105 has length and width dimensions of about three inches by about two to three inches, respectively, and a depth of about one to two inches. Other embodiments of case 105 may include larger and/or smaller dimensions. A depth dimension is selected, in one embodiment, to hold multiple (e.g., several) medicaments.

Case 105 includes, in this embodiment, compartment 110 accessible through partially or completely detachable door 112 on the front portion of case 105 (e.g., a partially detachable door may have one or more hinges that are connected to each of door 112 and case 105). FIG. 2 shows a front view of medicament container 100 with door 112 partially open to show compartment 110. As viewed, compartment 110 is a single, hollow area defined by case 105 suitable for holding multiple medicaments. In one embodiment, compartment 110 is configured to allow various medicaments to be co-mingled within compartment 110 without the need of sorting the various medicaments into different places. In this embodiment, the various medicaments may be randomly placed within compartment 110.

In the embodiment shown in FIG. 1 and FIG. 2, medicament container 100 is light weight and relatively small so that it is easy for a person to transport. FIG. 3 shows an embodiment of a medicament container attached to the belt of a person. In one embodiment, medicament container 100 includes clip 115 (see FIG. 1), such as a plastic body mounted at one end to a back portion of case 105 and extending longitudinally in a cantilever fashion, such that a patient may attach clip 115, for example, to a person's belt.

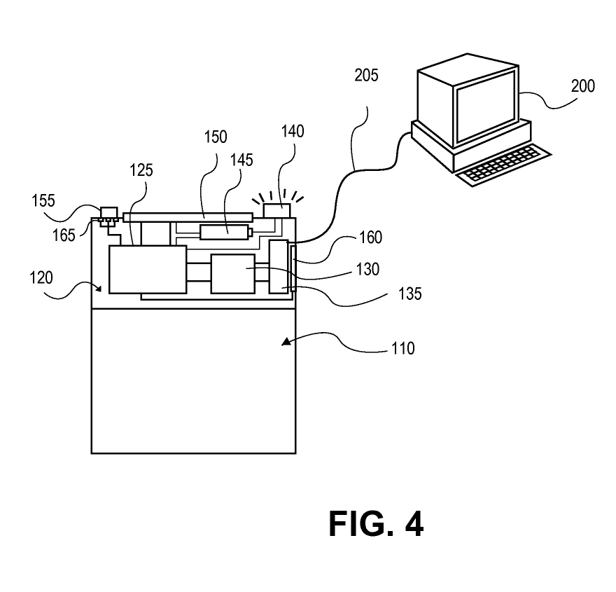

Referring again to FIG. 1 and FIG. 2, medicament container 100 also includes, in this embodiment, display 150 on a top portion of case 105. A top portion of case 105 also includes, in this embodiment, indicator 140, switch 155, and audio receiver 165. One side portion of case 105 includes, in this embodiment, interface 135 and audible output device 160. Details about display 150, indicator 140, switch 155, audio receiver 165, interface 135, and audible output device 160 are discussed with reference to FIG. 4.

FIG. 4 shows a cross-sectional side view of case 105 through line A-A′ of FIG. 1. Case 105 includes compartment 110 having dimensions suitable to hold multiple medicaments. In one embodiment, case 105 also includes interior space 120 containing processor 125, memory 130, and power source 145. Interior space 120 is shown separated from compartment 110 (e.g., by an internal wall).

Processor 125 in the embodiment shown in FIG. 4 is connected to memory 130 and power source 145. In one embodiment, processor 125 may be a microprocessor, including a dedicated uniquely designed architecture processor, or any other suitable microprocessor or microcomputer. Thus, processor 125 can be a processor sufficient to execute computer software embodied in a computer program product.

Referring to FIG. 4, processor 125 is connected (e.g., electrically connected) to memory 130. In one embodiment, memory 130 is of a size suitable to store medicament information data (e.g., name, dosage, descriptions, etc.) as well other data (e.g., time, date, patient instructions, etc.). In another embodiment, memory 130 is of a size suitable to store program instructions for displaying medicament data as well as other data to be displayed to the user.

Power source 145 is connected to and drives processor 125. Power source 145, in one embodiment, is a high energy density cell or plurality of cells. For example, power source 145 may be a battery (e.g. AA or AAA battery), a button cell battery, a coin cell battery, a rechargeable battery or any other power source suitable for use in a portable computing device.

As noted above, in the embodiment described with reference to FIG. 4, medicament container 100 includes indicator 140 connected to processor 125. Indicator 140 may also be connected to power source 145. Indicator 140 is located on a top side of case 105. For example, indicator 140 may be an audible alert (e.g. audible alarm), visual alert, or both. Representatively, FIG. 4 shows indicator 140 as a light such as a light emitting diode (LED) that may be energized by processor 125 at a time required to take a particular medicament. In another embodiment, indicator 140 is a vibration alert, representatively connected to processor 125 and positioned, for example, on a back portion of case 105. Processor 125 may energize indicator 140 to cause case 105 to vibrate.

Referring to the embodiment illustrated in FIG. 4, processor 125 is also connected to display 150 and display 150 may be connected to power source 145. Display 150 is of a size suitable to convey readable information to a person (e.g., patient) holding or wearing case 105. In one embodiment, display 150 is a liquid crystal display (LCD) screen. Display 150 may also be, for example, a plasma screen or any other electronically controlled screen. Processor 125 sends signals to display 150 outputted as readable information.

In the embodiment illustrated in FIG. 4, processor 125 is also connected to audible output device 160, such as an audio speaker. Audible output device 160 may include a digital to analog converter and other circuitry capable of receiving signals from processor 125 and converting the signals into audible information such as verbal instructions. Audible output device 160 may be a device capable of making audible at least some portion of the information stored in memory 130 and executed by processor 125, such as some portion of the information shown on display 150. This audible information may be presented along with or instead of information shown on display 150. In one embodiment, audible output device 160 may also sound an alarm to alert the patient it is time to take one or more scheduled medicaments.

Referring to the embodiment illustrated in FIG. 4, medicament container 100 includes switch 155 on a top side of case 105. Switch 155 is also connected to processor 125. Switch 155 may be, for example, a button. Other suitable switches include, but are not limited to, dials, knobs or any mechanism that can be moved or actuated by a person to trigger processor 125. Representatively, switch 155 may trigger processor 125 to read memory 130 and send a signal to display 150 to output readable information or send a signal to audible output device 160 to generate audible information from medicament container 100. In another embodiment, medicament container 100 contains more than one switch 155 capable of changing the information displayed on display 150.

In the embodiment illustrated in FIG. 4, medicament container includes audio receiver 165 such as a microphone connected to processor 125. Audio receiver 165 may include an analog to digital converter and other circuitry capable of receiving verbal (audible) input and converting the input into signals that may be read by speech recognition software contained in processor 125. Instructions may be sent to processor 125 manually via switch 155 or audibly via audio receiver 165.

FIG. 4 representatively shows computing device 200, such as a personal computer. Computing device 200 may be any device capable of receiving, storing and transferring data. Computing device 200 is independent of medicament container 100 and, in one embodiment, may be electronically connected to medicament container 100 via interface 135 (e.g., cable 205 is connected at one end to computing device 200 and at another end to interface 135). Data is transferred from computing device 200 through interface 135 into memory 130. Although illustrated as cable 205, the data may be transferred remotely using a radio frequency or other wireless transmission.

Computing device 200 may receive, store and transfer medicament schedule and description data. With regard to description data, medicaments are not generally manufactured with the name of the medicament printed on it. Instead, this information is typically printed on the label of the packaging (e.g. pill bottle) with which the medicament is distributed to a user (e.g., a patient or a patient's caregiver). Therefore, it is difficult to know exactly what a particular medicament is by inspection of the medicament itself. However, medicaments include a variety of characteristics that make them readily identifiable provided a user has the knowledge to distinguish particular medicaments based on these characteristics.

Medicament characteristics include, but are not limited to, size; shape; color; colors; flavor; scent; texture; visual marks, for example, letters, numbers, cross-top, lines, indentations, palpable marks, corrugations, etc.; type of medicament, for example, a description whether the medicament is a tablet, capsule, gel capsule, pill, liquid, powder, spray, lotion, cream, ointment, etc; or other description capable of being detected by at least one of the five senses. Knowing one or more of these characteristics allows a patient to distinguish medicaments from one another.

Referring to FIG. 4, in one embodiment, computing device 200 may be programmed by a healthcare professional to include schedule data and appropriate medicament description data, including one or more medicament characteristics. The data may be inputted directly into computing device 200 by the health care professional or inputted in response to questions asked by a program stored in computing device 200. Representatively, computing device 200 has a capability to read, compile or store a resource such as The Physicians Desk Reference®. This allows a healthcare professional to access medicament information from the resource and apply it to the programming of computing device 200.

In one embodiment, after the medicament and schedule data is input into computing device 200, the health care professional transfers the data to memory 130 of medicament container 100 via interface 135. Interface 135 includes an external receiving end sufficient to receive data from magnetic or optical disks, cards, wires, wireless signals, etc. external to case 105. This information may also be saved on a hard drive of computing device 200 or to an external storage device, such as a floppy disk or compact disc that may be placed into a patient's file and/or given to a person responsible for medicament container 100 for their records or reference.

Referring to the embodiment of medicament container 100 illustrated in FIG. 4, processor 125 is programmed to execute machine-readable instructions using the data transferred from computing device 200. In one embodiment, the machine-readable instructions are stored in a read only memory (e.g., an electronically programmable read only memory) in medicament container 100. Alternatively, the machine-readable instructions may be carried out by specific hardware components, or by any combination of programmed and hardware components. For example, the instructions may be part of an application specific integrated circuit (ASIC) or some combination of programmed components and an ASIC.

The machine-readable instructions in processor 125 include instructions to convey schedule data and medicament description data (e.g., dose and description of one or more characteristics of the medicament(s) a person is scheduled to take or administer at a particular time). An internal clock, in one embodiment, is included as part of processor 125 to be checked against to indicate, for example, when a medicament is scheduled to be taken. In one embodiment, processor 125 executes the instructions conveying the name, dose, and description of one or more characteristics of the medicament(s) against the clock and the executed results are shown on display 150. Processor 125 may also signal audible output device 160 to sound an audible alert and/or generate verbal instructions.

The following paragraphs describe methods of using medicament container 100. It is appreciated that the methods are presented as examples of such use rather than limits on the possible uses of medicament container 100.

In one embodiment, medicament information is transmitted from computing device 200 to medicament container 100 via interface 135 and is stored in memory 130. A person (e.g., a patient) or healthcare professional loads (stores) one or more medicaments in compartment 110. Utilizing an internal clock, at an appropriate time to take or administer a medicament (administration alert time), processor 125 triggers indicator 140 to alert the person having control of medicament container 100. Processor 125 may alternatively or additionally signal speaker 160 to audibly alert the person.

At this administration alert time, processor 125 also executes instructions from memory 130 regarding a particular medicament stored in compartment 110. Such instructions are displayed on display 150 and include, for example, the time a medicament is to be taken, the medicament name, dosage, one or more particular characteristics about the medicament, and any cautionary or other information regarding the medicament. In another embodiment, processor 125 may alternatively or additionally signal speaker 160 to audibly instruct the person as to some or all of the above information.

In one embodiment, at the administration alert time, the above information is automatically shown on display 150 or communicated through audible output device 160 without a prompt from the person using medicament container 100. In another embodiment, after an alert by indicator 140 and/or audible output device 160, moving or actuating switch 155 is necessary to access the above instructions from display 150 and/or audible output device 160. Additional moving or actuation of switch 155 may be necessary to obtain additional medicament information, such as “take pill with two glasses of water” or “pill may cause drowsiness, do not drive.”

Following the administration alert, a user (e.g., a patient or a patient's caregiver) opens door 112 of case 105 and accesses one or more medicaments within compartment 110 based on the information shown on display 150 or communicated through audible output device 160. In the embodiment where compartment 110 is one undivided or indivisible compartment, the information (e.g., one or more medicament characteristics) allows a user to differentiate between different types of medicaments that may be in compartment 110.

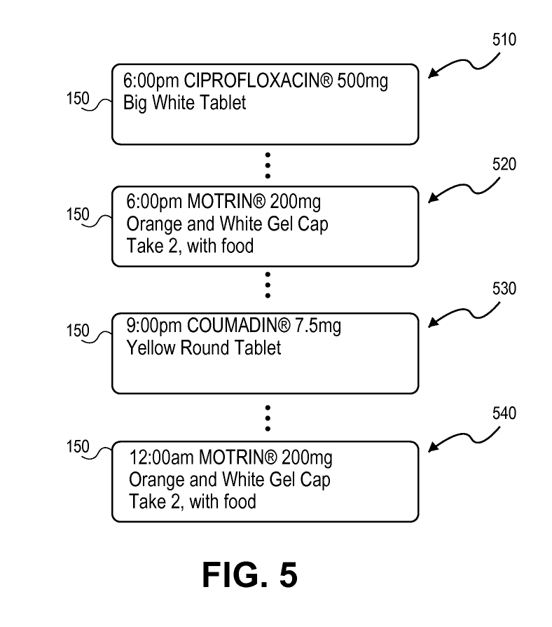

FIG. 5 shows a series of screen iterations of display 150 representing, for example, a schedule of medicaments to be taken for a portion of one day. These iterations may be automatically displayed at the appropriate administration alert time or prompted by moving or actuating switch 155. In one embodiment, iteration 510 shows the scheduled administration alert time, name, dosage, and one or more characteristics of a scheduled medicament. In another embodiment, multiple iterations may be necessary to show the complete information. For a particular administration alert time, multiple iterations may be necessary to display multiple medicaments (e.g., iteration 510 and iteration 520). These multiple iterations may need to be prompted by switch 155.

FIG. 5 also shows iteration 530 and iteration 540 representing medicament information for administration times subsequent to iteration 510 and iteration 520. Iteration 540 is an administration time following an administration time represented by iteration 530.

In between administration alert times, a user may be able to access the next or previously taken medicament information by, for example, moving or actuating switch 155. In another embodiment, a plurality of switches 155 may be utilized to access medicament and/or schedule information.

In another embodiment, processor 125 may be accessed by voice commands through audio receiver 165 to perform all functions previously described for switch 155. Audio receiver 165 may be used additionally or alternatively to switch 155.

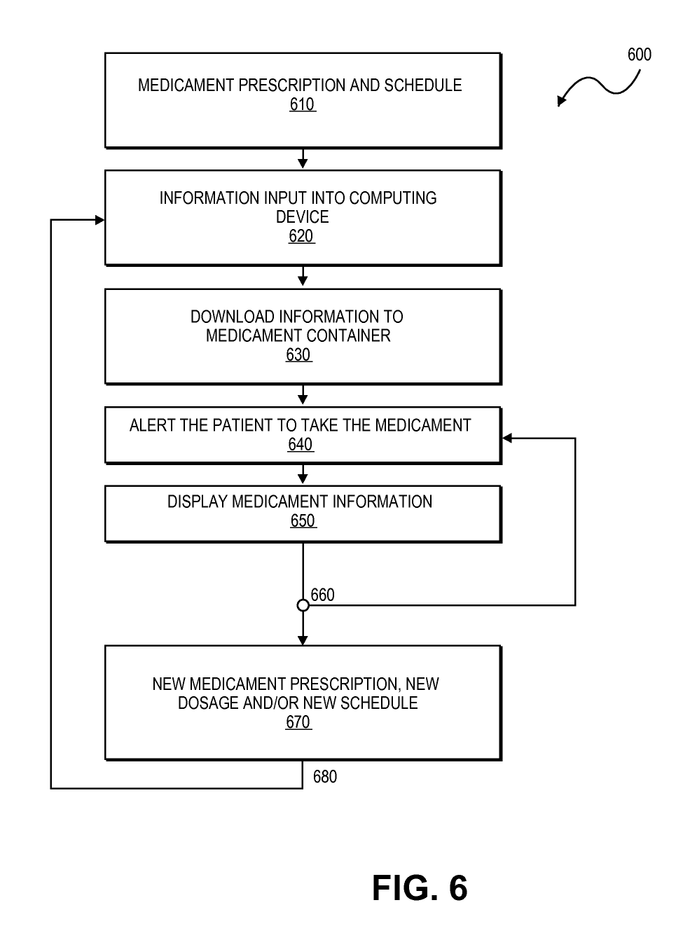

FIG. 6 shows a block diagram of a method of reminding a user (e.g., a patient or a patient's caregiver) to take or administer a medicament. In this embodiment, method 600 begins with a healthcare professional prescribing medicament(s) and the schedule of when the user is to take or administer the medicament(s) (block 610). The healthcare professional, in one embodiment, may then input the schedule, medicament names, dosages and medicament characteristic(s) into an independent computing device (block 620). In one embodiment, the independent computing device contains a copy of The Physicians Desk Reference or other material for reference.

In one embodiment, the times, medicament names, doses, characteristics and other information are programmed or downloaded from the independent computing device via a cable link or wireless link into a memory executable by a processor contained within the medicament container (block 630).

When a user is scheduled to take or administer a medicament, the user is alerted (block 640). In one embodiment, when the alert is given, the time, medicament name, dose, at least one characteristic of the scheduled medicament and other information is described to the patient (block 650). The user is subsequently alerted, in one embodiment, of additional medicaments to take or administer at the same time, if any, or future scheduled times to take or administer the medicament, and the appropriate descriptions of the medicament (660).

The above method may continue for some time such as for a length of a prescription period of one or more medicaments. After such time, a health care professional may evaluate, for example, a particular treatment regimen and update the regimen. For example, a health care professional may prescribe a new medicament, a new medicament dosage, and/or a new schedule (block 670). At that point, the health care professional would input the new information into an independent computing device and ultimately to the medicament container (680).

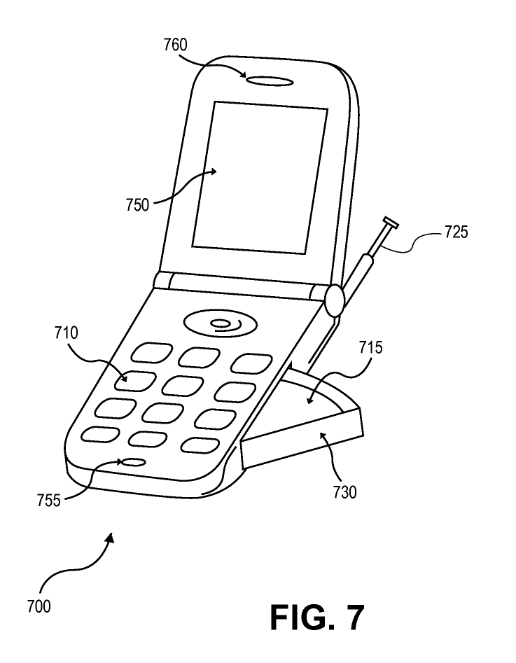

FIG. 7 shows another embodiment. This embodiment may combine one or more of the features discussed above with a mobile computing and communication platform allowing wireless communication between a healthcare professional and a user such as a patient or the patient's caregiver. Communication and computation apparatus 700 bears some resemblance to a cellular telephone, but embodiments can also be constructed by leveraging the computing and communication facilities present in a device such as a personal digital assistant (“PDA”) or a pager.

External features of this embodiment of mobile communication and computation apparatus 700 that are visible in FIG. 7 include display 750, audible output device (speaker) 760, keypad 710 and microphone 755. Mobile communication and computation apparatus 700 also includes medicament compartment 715. In this embodiment, medicament compartment 715 is defined by hinged side drawer 730 that is shown in its open position, revealing medicament compartment 715. Such a compartment may be connected to the base or back of a standard mobile phone, for example, by a snap-on or force fitting. In some embodiments, multiple separate medicament compartments may be provided. Compartments may be opened under the control of logic in the device, so that only one of several compartments opens at a particular time. For example, hinged side drawer 730 of compartment 715 may have a magnetic closure mechanism where, for example, a first strip of magnetic material is attached to a leading edge of hinged side drawer 730 and a separate strip is connected to a body of communication and computation apparatus 700. Circuitry in communication and computation apparatus 700 is connected to the second strip and may change the polarity of the second strip to one opposite the polarity of the first strip (to keep hinged side drawer 730 closed) and to one similar to the polarity of the first strip to allow hinged side drawer 730 to be opened. This feature may be useful to prevent user (e.g., patient) confusion between medicaments of similar appearance: the device opens only the compartment containing the correct medicament when scheduling logic indicates that it is time to use the medicament.

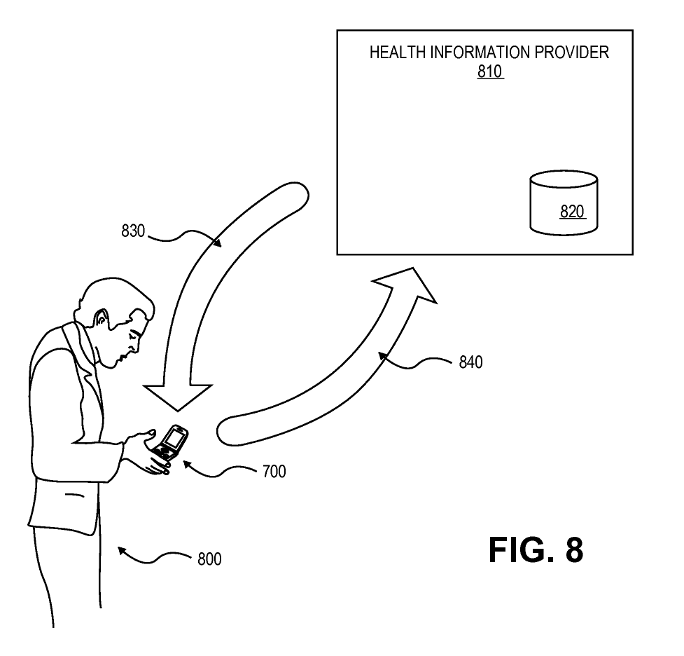

Communication and computation apparatus 700 shown in FIG. 7 operates as part of a larger health care system environment, an example of which is shown in FIG. 8. A user (e.g., patient 800) carries mobile communication and computation apparatus 700 such as a mobile phone or PDA. Representatively, communication and computation apparatus 700 may contain a SIM card for a GSM network (radio frequency). Communication and computation apparatus 700 also includes antenna 725 to transmit and receive signals. Communication and computation apparatus 700 may support voice calls and may further support text messaging, for example, short message service (SMS); access wireless application protocol (WAP) to enable internet access; and multimedia messaging service (MMS) to enable the sending and receipt of multimedia objects such as images, audio, video, rich text, etc. In another embodiment, communication and computation apparatus 700 includes a global positioning system (GPS) to, for example, aid in dispatching emergency responders.

Referring to FIG. 7, one or more medicaments are stored in communication and computation apparatus 700, although they are not shown in this figure. The one or more medicaments may be placed in the apparatus by a patient himself, or by a caregiver, a nurse, a pharmacist, or other healthcare professional. In another embodiment, mobile communication and computation apparatus 700 does not contain a storage capability for storing one or more medicaments. Instead, mobile communication and computation apparatus 700 may be used in conjunction with a separate portable medicament container, such as medicament container 100 described above with reference to FIGS. 1-6 and the accompanying text, or used without a portable medicament container at all, for example in a situation where a user (e.g., a patient or a patient's caregiver) stores the one or more medicaments in an alternate location such as in a purse, briefcase, bag, pocket or at home in a doctor- or pharmacy-supplied container.

Referring to FIG. 8, in one embodiment, health information provider 810 (for example, a hospital, a doctor's office, or a pharmacy) uses information about a patient stored in database 820 to determine when to contact a user such as patient 800 regarding the patient's medicaments. For example, database 820 may store, among other things, information about a patient's health and prescription(s) and a time to take a particular medicament (e.g., medicament A, three times a day with a meal, at 7:00 a.m., 12:00 noon and 6:00 p.m.). When a time approaches or arrives for the patient to use a medicament stored in communication and computation apparatus 700 or elsewhere, health information provider 810 activates the communication functions of communication and computation apparatus 700 by placing a telephone call over a cellular telephone network, sending a text message, or some similar means (830). This may be done manually by a doctor, pharmacist, or an assistant triggering the communication, or automatically by a computer system programmed to trigger the communication, at a particular time.

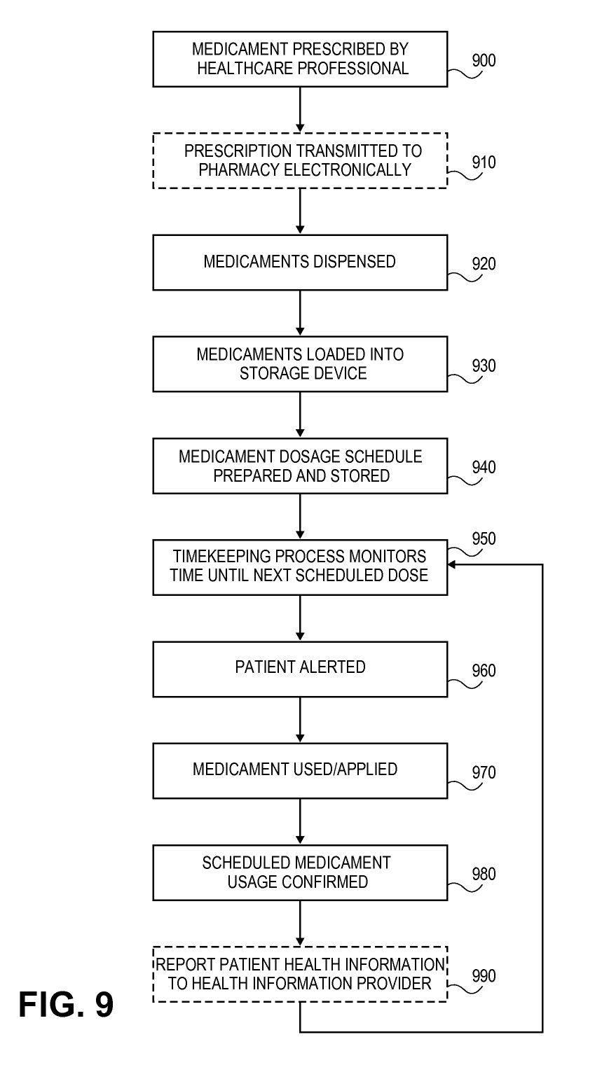

Automated notifications of medicament usage schedules can be implemented according to the method outlined by the flow chart in FIG. 9. First, a doctor, physician or other healthcare provider prescribes a medicament or medicaments for a patient (block 900). The prescription may be given to a user (e.g., a patient or a patient's caregiver) as a traditional paper record, but some embodiments may transmit the prescription details electronically to a pharmacy, as shown in phantom block 910. A pharmacist dispenses the prescribed medicaments (block 920), and the medicaments are optionally loaded into a storage device used with an embodiment (block 930). Medicament loading may be performed by theuser, by the pharmacist, or by another healthcare professional. In some embodiments, medicaments may be dispensed into a mobile communication and computation apparatus such as communication and computation apparatus 700 with medicament compartment 715 (see FIG. 7). Alternatively, medicaments may be dispensed into a disposable medicament container that can be attached to a communication and computation apparatus via snap-on clips or similar means. In other words, instead of receiving a plastic bottle containing medicaments which must be transferred to another medicament container for use with an embodiment, a user (e.g., a patient or a patient's caregiver) may receive a pre-loaded medicament container that can be connected or inserted into a device that performs the communication, data collection and information display, timekeeping and other functions according to an embodiment. When the medicament in the pre-loaded medicament container has been used, the medicament container may be disconnected from the apparatus and discarded or returned to a pharmacy. Alternatively, medicaments may be dispensed into conventional containers and the user may place the medicaments or container in a place of his/her choosing.

Before, during or after the time the prescribed medicaments are dispensed, a dosage schedule is prepared and stored (block 940). The schedule may be prepared based on information about the medicament stored in the pharmacist's computer, special instructions from the physician communicated with the prescription, and other information provided by the patient. The schedule may be transmitted along with other information (e.g., medicament characteristic(s), warnings, etc.) (downloaded) to a mobile communication and computation apparatus, stored in a database at the pharmacy, the physician's office or hospital, or another accessible location.

Subsequently, throughout the course of the patient's treatment with the medicament, an automatic timekeeping process tracks the time of the next scheduled medicament use (block 950). Timekeeping may refer to an absolute (wall clock) time, or may track an elapsed time so that a scheduled dose occurs at a predetermined time after a previous scheduled dose. The automatic timekeeping process may be performed by computation logic within the mobile communication and computation apparatus, or by a computer system located remotely from the mobile communication and computation apparatus (for example, a computer at the pharmacy or healthcare provider). When the scheduled medicament usage time arrives, a user of a mobile communication and computation apparatus (e.g., a patient, or a patient's caregiver) is alerted (block 960) via a light, sound, vibration or other signal to the mobile communication and computation apparatus. Where the mobile communication and computation apparatus contains a drawer or container holding a medicament, the drawer or container holding the medicament scheduled to be used can be opened, or where multiple different medicaments may be in the drawer or container (possibly commingled) or no drawer or container is associated with mobile communication and computation apparatus, an image or description of the correct medicament can be presented to the user via the mobile communication and computation apparatus. For example, a message alert may appear on a liquid crystal display (LCD) associated with the mobile communication and computation apparatus that describes in text or image form medicament characteristic(s), as well as any warnings or other alerts (e.g., “don't drive,” “take with a meal,” etc.). If the automatic timekeeping process is performed by a remote system, the alert can be transmitted to the medicament container via a cellular telephone or other wireless data network as a voice message or text message.

In one embodiment, the user uses or applies the medicament (block 970) and interacts with the mobile communication and computation apparatus to confirm that the scheduled usage has occurred (block 980) by, for example, calling or text messaging a pharmacy or healthcare provider. Alternatively, confirmation can be sent automatically. The mobile communication and computation apparatus may use its communication facilities to contact and report to the healthcare provider or pharmacist at this time (block 990). In an embodiment where a scheduled dose occurs at a predetermined time after a previously scheduled dose, the report to the healthcare provider or pharmacist that a scheduled dosage (usage) has occurred may trigger the timekeeping process. Alternatively, the report to the healthcare provider or pharmacist may be stored as historical information about a patient and a particular medicament in connection with a treatment plan. Alternatively, information may be stored for later manual retrieval.

In addition to allowing reporting of a scheduled dosage, some embodiments of a mobile communication and computation apparatus can collect general patient health information such as one or more of blood pressure, pulse, glucose levels, or the like. This information can be reported to the healthcare provider so that the progress of the patient's treatment plan can be evaluated or be retrieved in case of an emergency. For example, a mobile communication and computation apparatus may be equipped with sensors such as a thermometer, blood pressure or glucose level meter, pulse, heart auscultation device, or respiration sensor.

In addition to notifying a user (e.g., a patient or a patient's caregiver) of a schedule to take or apply a particular medicament, medicament characteristic(s) and any warnings associated with a medicament or a treatment regimen, the user may also be notified of other medical or health information. Such other information includes, but is not limited to, doctor's appointment reminders, ancillary medical reminders (e.g., reminders to attend physical therapy), and other medical or health related reminders, such as a presurgical reminder not to consume food.

The operations described in the method with reference to FIG. 9 (timekeeping, alerting, medicament usage, confirming, and reporting) are repeated throughout the course of the patient's treatment. In another embodiment, a method includes one or more but not each of the operations described with reference to FIG. 9. For example, in one embodiment, a method may include the operations of timekeeping, and alerting without the operations of a user (e.g., a patient or a patient's caregiver) confirming or reporting medicament usage or application. In this embodiment, confirmatory information about the patient's medicament usage or application and possibly general patient information may be stored in an apparatus such as a mobile communication and computation apparatus so that a physician or other healthcare provider can retrieve and view the information at a next check-up.

Returning to FIG. 8, information may be transmitted to mobile communication and computation apparatus 700 as indicated by arrow 830 via a cellular network, a wireless data connection, an optical channel, or other similar data carrier. This information may simply remind a user such as patient 800 to use a medicament, or it may provide detailed instructions and/or a picture of the medicament. Transmitted information may be in the form of a voice mail message, an SMS, an MMS, a picture or graphic image, an animated sequence or video, or a combination of such elements.

In some systems, health information provider 810 can use the data communication capabilities of mobile communication and computation apparatus 700 to download commands and information to the mobile communication and computation apparatus at a first time, for later presentation or execution by the apparatus. Thus, the system can arrange for scheduled reminders to a user (e.g., a patient or a patient's caregiver) even when the mobile communication and computation apparatus cannot be reached (e.g., its communication functions are turned off or it is out of range). In such an embodiment, mobile communication and computation apparatus 700 may include a timekeeping subsystem such as a clock, or may obtain the current time from an external source such as a radio broadcast.

In one embodiment, a user such as patient 800 in FIG. 8 may initiate a data connection with health information provider using the communication capabilities of mobile communication and computation apparatus 700. Such a data connection may be used to notify the health information that patient 800 has taken the prescribed medicament at the appointed time. Additionally, the data connection between the patient and the health information provider may also be used by patient 800 to obtain additional details about a medicament (e.g., additional physical characteristics, warnings, etc.), or to alert the health information provider of an unexpected side effect of the medicament.

In other embodiments, a mobile communication and computation apparatus may be equipped with sensors such as a thermometer, blood pressure or glucose level meter, pulse, heart auscultation device, or respiration sensor. These sensors may collect information about the patient's health to be relayed back to health information provider 810, as shown by arrow 840. Health information provider 810 can use this information to adjust a medicament dosage schedule or to call for emergency medical assistance for patient 800.

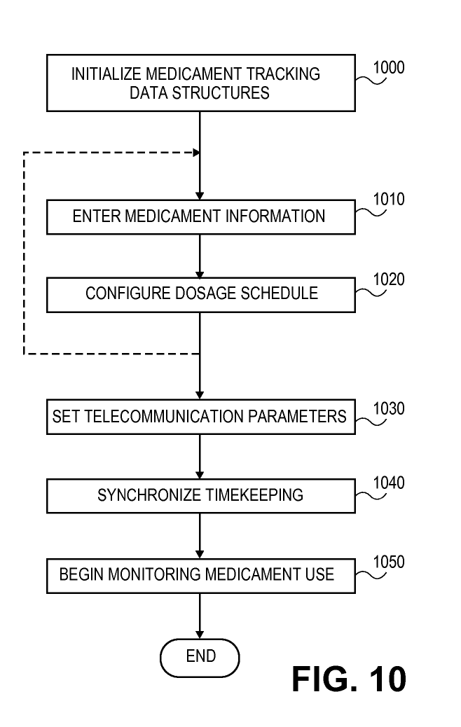

FIG. 10 outlines actions that may occur to prepare a healthcare provider's system to work with an embodiment of a mobile communication and computation apparatus. First, a set of data structures for tracking medicament schedules and use are initialized (block 1000). Then, for each medicament, information about the medicament is entered (block 1010) and a dosage schedule for the medicament is configured (block 1020). After all the medicament and dosage schedule information has been set, telecommunications parameters are configured (block 1030). For example, the system may be configured to use a wireless Internet Protocol (“IP”) network to communicate with the medicament container, or a cellular telephone system or text-based message service (e.g. SMS, MMS) may be selected. The system's timekeeping functions (e.g. clocks) are synchronized (block 1040) and the system can begin monitoring medicament use (block 1050).

Some embodiments can be performed by software executing on a general- or special-purpose processor. Thus, an embodiment can be executable instructions stored on a computer-readable medium, the instructions to cause a programmable processor to perform operations as described above. A computer-readable medium may be a read-only memory (“ROM”) cartridge, a Flash memory cartridge, a compact disk read-only memory (“CD-ROM”), a magnetic medium such as a floppy disk, or a transmission of instructions across a data communication network such as the Internet. Executable instructions may be stored as binary data that are directly executable by a processor, or as human-readable “source code” that can be processed by automatic tools such as compilers and linkers to prepare instructions suitable for machine execution.

In the above embodiments, a user (e.g., a patient or patient's caregiver) interacts with the patient's physician, the physician's office, pharmacist or other medical professional to coordinate, among other things, a schedule for using or applying a medicament; a reminder system for notifying the user to use or apply a particular medicament or medicaments; and an identification of a medicament or medicaments (medicament characteristic(s)) to be taken; doctor's appointment reminders; other ancillary medical reminders (e.g., physical therapy); medical test appointment reminders; and other medical or health related reminders, such as “don't eat 8 hours before surgery”. In another embodiment, a user may establish one or more of these features independently, without the assistance of a medical professional. One situation where independent establishment of medicament information including, but not limited to, dosage and medicament characteristic(s) may be utilized is where long term or chronic administration of a medicament is necessary and a medical professional's continuous reminding or monitoring may be unnecessary.

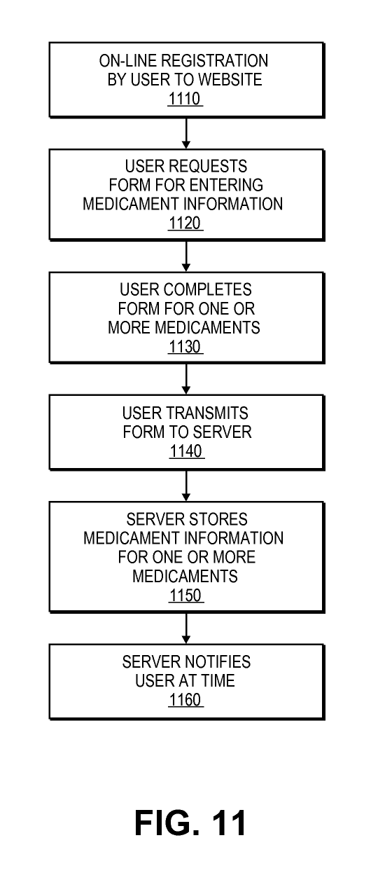

FIG. 11 shows one representative method where a user (e.g., a patient or a patient's caregiver) interacts with a medicament communication facility to establish a notifying system for taking or using one or more medicaments. Referring to FIG. 11, a communication facility may establish a website on a server that may be transmitted over the internet and can be accessed through a web browser. The website may permit a user to register at the communication facility server (block 1110). The user may then request a form from the server to enter a particular medicament or medicaments along with a dosage schedule, medicament characteristics of the medicament or medicaments and/or any other information (e.g., warnings) (block 1120). The user completes the requested form providing medicament information (e.g., medicament characteristic(s) and other medical or health related information). The user also specifies a preferred notification system for the user to be notified by the communication facility, such as via voicemail message, text message, etc. and the information associated with that reminder system (e.g., phone number, email address, etc.) (block 1130). The user then transmits the form through the user's web browser to the server (block 1140).

In another embodiment, the communication facility may query the user about one or more medicaments that the user may be administering. Such query may be, for example, the type of medicament relative to treatment (e.g., heart medicine) or brand name. In response to an answer or answers transmitted by the user, the communication facility may present (through the user's browser) one or more possible medicaments and queries the user a second time to select a prescribed medicament. The user then selects the medicament from the one or more possible medicaments and the selection is provided to the communication facility. The communication facility may then seek out, either from its library or from the medicament manufacturer or other source, medicament characteristic(s) of the medicament and/or any other information without requiring this information from the user.

The information provided by a user and sent to the communication facility's web server is stored (block 1150). The communication facility then notifies the user by the user's preferred reminder system at time intervals consistent with the dosage schedule (block 1160). For example, the communication facility may notify a user through the user's home telephone, email, cell phone, mobile communication and computation apparatus (e.g., communication and computation apparatus 700, see FIG. 7) or other communication device. The notifying may be done by a live person or automatically (e.g., by voice recordings from a processor at the central station, text messages, etc.) The notifying may alert the patient to take a medicament, provide medicament characteristic information, other medicament information (e.g., warnings), and/or medical or health related information or reminders (e.g., appointments, physical therapy, etc.). In one embodiment, the communication facility may charge the user for each notification or offer a periodic (e.g., monthly, annually) subscription in which the user pays a single fee and is notified with the appropriate medicament information throughout a period.

In another embodiment, a communication facility may provide, possibly for a fee, notification software that may be downloaded onto a user's personal computer or any communication and computation apparatus such as, but not limited to a cell phone or mobile communication and computation apparatus 700. The downloaded software may convert a cell phone into an apparatus that performs one, multiple or all the functions described heretofore such as, but not limited to, allowing the user to establish a schedule for using or applying a medicament or medicaments; a reminder system for notifying the user to use or apply the medicament(s); an identification of the medicament(s) (e.g., medicament characteristic(s)); and/or other medical or health information including but not limited to other reminders. The downloaded software may also configure a cell phone or communication and computation apparatus to measure and/or gather patient health information such as blood pressure, pulse, and glucose level (in one embodiment, in connection with one or more peripheral devices). The software once downloaded on a user's personal computer, cell phone or communication and computation apparatus may gather the above-referenced information in a manner similar to the manner a server maintained by a communication facility would gather the information, by requesting the user to complete a form. In this manner, a memory and a processor may be responsible for the functions described. This may be a memory and a processor associated with a user's cell phone or mobile communication and computation apparatus or a user's personal computer (e.g., home computer) programmed through the notification software to notify the user's home telephone, email, cell phone, mobile communication and computation apparatus or other communication device.

In the preceding paragraphs, specific embodiments are described. It will, however, be evident that various modifications and changes may be made thereto without departing from the broader spirit and scope of the claims. The specification and drawings are, accordingly, to be regarded in an illustrative rather than a restrictive sense.

I claim:

1. An apparatus comprising:
a compartment to store a medicament;
a communication facility to exchange data with a health information provider;
a display system to present information to a user; and
a timekeeping subsystem to activate the display system at a predetermined time.

2. The apparatus of claim 1, further comprising:
a sensor to measure health information of a patient.

3. The apparatus of claim 1 wherein the communication facility uses a cellular telephone network.

4. The apparatus of claim 1 wherein the communication facility uses a Short Message Service (“SMS”) network.

5. The apparatus of claim 1 wherein the compartment is pre-loaded with the medicament.

6. The apparatus of claim 1 wherein the compartment is adapted to be removably coupled to the apparatus.

7. A method comprising:
transmitting a first message to a mobile device, the first message to alert a user of a scheduled administration of a medicament; and
receiving a second message from the mobile device to confirm that the user has administered the medicament.

8. The method of claim 7 wherein transmitting comprises communicating with the mobile device via a cellular telephone network.

9. The method of claim 7, further comprising:
receiving a message from the mobile device, the message to contain health information about a patient.

10. The method of claim 7, further comprising:
transmitting at least one medicament characteristic about the medicament to the mobile device.

11. A computer-readable medium containing executable instructions to cause a programmable processor to perform operations comprising:
alerting a user of a scheduled administration of a medicament and at least one medicament characteristic of the medicament.

12. The computer-readable medium of claim 11, containing additional executable instructions to cause the programmable processor to perform operations comprising:
receiving a message from a health information provider via a communication facility; and
alerting in response to the message.

13. The computer-readable medium of claim 11, containing additional executable instructions to cause the programmable processor to perform operations comprising:
collecting a measurement of health information of a patient; and
transmitting the measurement to the health information provider via the communication facility.

14. The computer-readable medium of claim 11, containing additional executable instructions to cause the programmable processor to perform operations comprising:
displaying the at least one medicament characteristic of the medicament on a display device.

15. The computer-readable medium of claim 11 wherein displaying the alert comprises playing a voice mail message.

16. The computer-readable medium of claim 11 wherein the alerting comprises sending a message over a mobile device network.

17. A method comprising:
gathering medicament information, the medicament information comprising a dosage schedule and at least one medicament characteristic; and
notifying of a scheduled administration of the medicament and at least one medicament characteristic.

18. The method of claim 17, wherein notifying comprises transmitting a message to a mobile device.

19. The method of claim 17, wherein gathering medicament information comprises:
accepting information from a user submitted through a browser.

20. The method of claim 17, wherein the gathering and notifying are performed through a mobile device.
